(19) United States
(12) Patent Application Publication
(10) Pub. No.: US 2022/0380751 A1
(43) Pub. Date: Dec. 1, 2022

(54) METHOD FOR ENRICHING MICROVESICLES

(71) Applicant: AJ Innuscreen GmbH, Berlin (DE)

(72) Inventor: Timo HILLEBRAND, Hoppegarten (DE)

(73) Assignee: AJ Innuscreen GmbH, Berlin (DE)

(21) Appl. No.: 17/664,119

(22) Filed: May 19, 2022

Related U.S. Application Data
(63) Continuation of application No. 15/321,689, filed on Jul. 11, 2017, now abandoned.

(30) Foreign Application Priority Data
Jun. 24, 2014 (DE) 10 2014 212 126.4

(51) Int. Cl. C12N 15/10

(52) U.S. Cl. CPC C12N 15/1003

(57) ABSTRACT

Microvesicles are enriched from a sample (for example exosomes) for subsequent isolation of biomolecules contained in the microvesicles, in particular RNA. A method involves: a) addition of an aqueous solution of salt of a polyuronic acid to the sample, b) addition of a substance which induces gel formation/pellet formation of the polyuronic acid, c) mixing of the sample and short incubation, d) centrifugation of the sample and removal of the supernatant, e) dissolving the pellet of gel piece, and 0 isolation of the biomolecules contained in the microvesicles, preferably RNA. Alginate is used as a preferred salt.

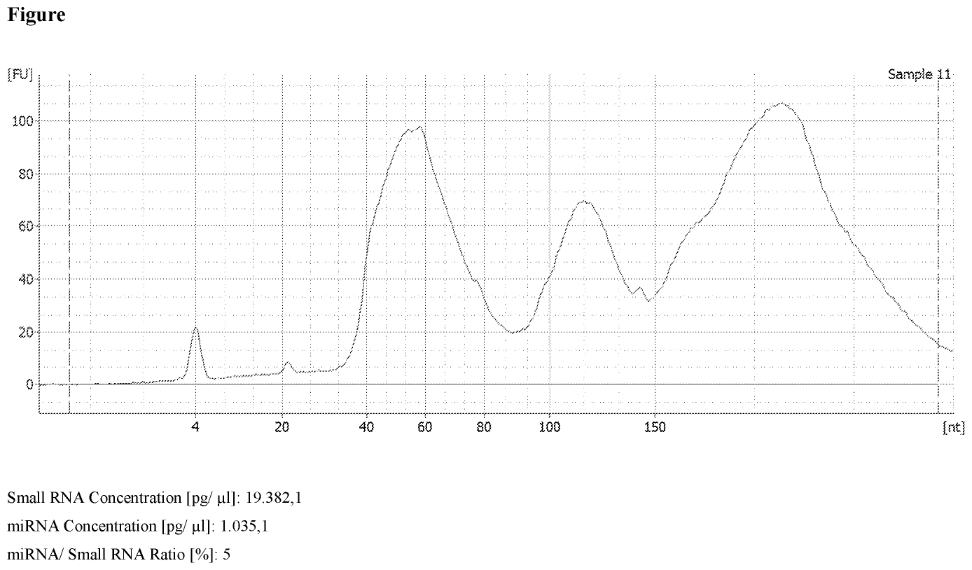

### CROSS-REFERENCE TO RELATED APPLICATIONS

This application is the continuation application of U.S. 15/321,689 filed on Jul. 11, 2017 which is a National Stage entry under 371 of International Application No. PCT/EP2015/064256 filed on Jun. 24, 2015 and which claims the benefit of priority to German application 10 2014 212 126.4 filed on Jun. 24, 2014. The contents of each of these applications is hereby incorporated by reference in its entirety.

### BACKGROUND OF THE INVENTION

### Field of the Invention

Subject matter of the invention is a simple method for enriching microvesicles from a sample (exosomes) for subsequent examination of said particles and/or for release and extraction of the nucleic acids entrapped in the exosomes, especially RNA.

### Description of Related Art

It is known that so-called freely circulating nucleic acids are located in cell-free body fluids which is not only DNA but also RNA which can be isolated from said samples. If the so-called cell-free circulating DNA is genuinely free DNA, it has to be assumed that the isolated RNA is no freely circulating RNA since RNAses occurring in body fluids would very rapidly cause a massive decomposition of free RNA. Therefore, it is assumed that the RNA is located in so-called exosomes.

Exosomes are vesicles which are given off by a cell to its environment. Their size is approx. 30 to 90 nm.

Exosomes develop in a multistage process and are finally given off to the cellular environment after a constriction of the cell membrane in a discharge process. The exosomes contain nucleic acids, especially RNA and proteins in varying composition. They serve as a transport vehicle, for the discharge of cellular components and according to recent findings above all for cellular communication. In this connection exosomes become increasingly important for the investigation of different diseases (cancer, virus infections, autoimmune diseases and much more). Of particular interest is here the investigation of miRNA and/or mRNA enclosed in exosomes because these nucleic acids by means of exosomes are transported from tumor cells in recipient cells and seem to be of vital importance for tumor growth. As important initial samples, for example, body fluids such as blood plasma or serum or urine are possible. But also other sample types are used for other aims of analysis (for example, milk samples). The problem is that the quantity of the exosomes existing in these samples generally is very low so that it would be desirable to process larger sample volumes.

Presently, only a few methods exist permitting to enrich and/or isolate exosomes from a sample and/or to subsequently extract the nucleic acids contained in the exosomes. Widely used is the utilization of ultracentrifugation techniques wherein exosomes are accumulated on the bottom of the reaction vessel. Said method is bound to an ultracentrifuge and moreover time-consuming and inappropriate for routine diagnostics. Moreover, also the technique of ultrafiltration is used. This method as well is time-consuming and very expensive. Alternative methods consist of an immunoprecipitation of the exosomes by means of an immunoplate or by means of immunobeads. This type of enrichment of exosomes is time-consuming as well and susceptible to faults due to the reagents to be used, and expensive. Moreover, only 200 to 500 μl of sample can be used in this technology.

The published patent application WO 2009/135936 A1 is also State of the Art. The subject matter of said publication is a simple method for the enrichment of biomolecules (for example, proteins or nucleic acids) from a sample for subsequent isolation of the biomolecules and sensitive detection, if applicable. The method comprises the following steps:

a) addition of an aqueous solution of a salt of a polyuronic acid to the sample
b) addition of a substance which induces gel formation/pellet formation of the polyuronic acid
c) mixing of the sample and short incubation
d) centrifugation of the sample and removal the supernatant
e) dissolving the pellet or the gel piece
f) isolation of the biomolecules in a manner known per se.
Alginate is used as a preferred salt.

Therefore, the isolation and enrichment of biomolecules by means of a salt of a polyuronic acid and subsequent gel formation is known in principle. Unknown, however, is that also exosomes can be enriched and isolated by said method.

### SUMMARY OF THE INVENTION

Therefore, it had been the task of the invention to permit enrichment of exosomes by means of a rapid and simple method in order to be able to work in particular also on large-volume samples. The method shall be compatible with a subsequent simple and rapid method for isolation of RNA located in the exosomes.

The task has been solved according to the features as described. The invention constitutes a selection invention, namely enrichment and isolation of the special biomolecules of the exosomes by means of a method which had already been known for enrichment and isolation of other biomolecules. But said invention is entirely surprising and is not suggested by knowledge of the known publication WO 2009/135936 A1. Although it is known to isolate nucleic acids, viruses or proteins in the manner described, it had been entirely surprising that exosomes can also be isolated by means of said method.

### BRIEF DESCRIPTION OF THE DRAWING

The Figure shows an electropherogram from which enrichment of the exosomes results.

### DETAILED DESCRIPTION OF THE INVENTION

Surprisingly it was found that certain polysaccharide derivatives can excellently be used for enrichment of exosomes in liquid samples with said enrichment step being compatible with an efficient method of isolation of the RNA enclosed in the exosomes. The polysaccharide derivatives are the salts of polyuronic acids. Particularly suitable are here the so-called alginates of the alginic acids. Alginates are structural elements in brown algae. Alginate is a polysaccharide which consists of 1.4 linked α-L guluronic acid (G) and β-D-mannuronic acid (M).

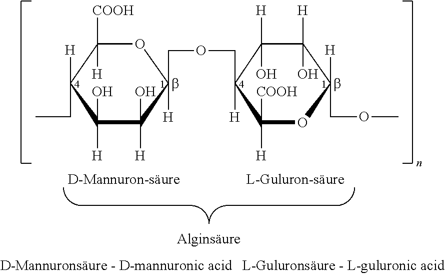

It forms homopolymeric areas in which mannuronic acid or guluronic acid exists as a block.

Alginates are used in the food, pharmaceutical and cosmetics industry, for example, as a gelling agent in the food industry, as a finishing agent for textiles, for the production of photographic papers or in dental practices for the production of dental and jaw impressions.

Alginate is also an important bio material. By encapsulation of human cell tissue with alginate it is possible to store exogeneous material, such as, for example, donor cells, without it being possible that they are recognized by the immune system, and destroyed. But there is no indication that alginates can be used for the enrichment of exosomes for a subsequent iso-lation and purification of enclosed RNA.

For the method according to the invention one uses the known ability of alginates to gel in solutions with low calcium content and to form so-called hydrogels. The cause of gelling is due to the storage of calcium ions into the zigzag structure of the GG-blocks. The zigzag structure of another alginate molecule then rests upon said zone. Three-dimensional structures form as a result. The formation of gels also occurs combined with strong acids. Moreover, the gel structures formed can also be specifically destroyed again.

By using the formation of alginate gels, enrichment of the exosomes for a subsequent molecular diagnostic analysis from a liquid sample, in particular from the difficult large volume samples, can be realized by means of the method according to the invention in a simple and rapid manner. In this connection, the method is safe regarding the use of chemicals and does not require any specialized equipment.

The method according to the invention for the enrichment of exosomes from a sample for subsequent isolation of RNA can, for example, take place as follows:

1. addition of an aqueous alginate solution to the sample
2. addition of an aqueous solution which induces gel formation/pellet formation (for example, using 1 M calcium chloride solution or a 1% hydrochloric solution)
3. mixing of the sample short incubation at room temperature
4. centrifugation of the sample and removal of the supernatant
5. dissolving the pellet or gel piece and subsequent isolation of the RNA in a manner known per se

The concentrations of alginate and reagent for gel formation used in the method according to the invention are very low. Consequently, the gelation process visibly does not take place as it is known from the food industry or as it is described for all applications of alginates. After centrifugation, the supernatant is removed. A small gel piece or pellet becomes visible on the bottom of the reaction vessel which gel piece/pellet contains the exosomes concentrated from the sample. Subsequently the gel piece/pellet is dissolved again by adding a solution which destroys the gel structure. In the last step, isolation of the RNA from the concentrated sample occurs by known methods. The method is extremely simple to realize. The incubation time and subsequent centrifugation can be completed within 5 to 10 minutes. Dissolution of the gel piece occurs easily. No ultracentrifugation is required so that one can work with normal standard table centrifuges. The volumes of the liquid initial samples can be freely chosen which permits a very broad range of applications. One can, for example, work with samples of 500 μl but also 10 ml which gives a huge range of applications.

By the method according to the invention, the initial sample volume is reduced to the gel piece/pellet formed in the process which can then be easily further processed in the so-called mini format of the RNA isolation.

For the method according to the invention, for example, one combines an aqueous alginate solution and an aqueous solution containing salts of divalent and/or polyvalent cations (for example, calcium chloride or aluminium chloride). Similarly, one can combine an aqueous alginate solution and a weak acid (hydrochloric acid). The destruction of the gel structure can be made by means of a buffer solution, which contains a chelating agent (EDTA) or by adding a solution of trisodium citrate dihydrate. But one can also dissolve the gel piece/pellet in a low-salt buffer (for example, 10 mM Tris-HCl) at an alkaline pH value.

A particularly efficient embodiment uses the observed effect that dissolution of the alginate gels developed is also excellently possible with buffers which are used for the isolation of RNA. In this process so-called chaotropic buffers, such as, for example, on the basis of guanidinium salts, destroy alginate gels in a general manner no matter by which process the alginate gel had been formed. Similarly, a form of RNA isolation known to the person skilled in the art can be used by utilizing a monophasic solution of guanidinium isothyocyanate/phenol for an RNA isolation downstream of the enrichment. The technical literature on alginates and their fields of application does not mentioned such an observed effect.

On the basis of this observation, the alginate gel can then be dissolved with a chaotropic buffer or a mixture of a chaotropic saline solution and phenol which subsequently are used at the same time for the process of isolation of the RNA with a dual function. In this preferred case, for example, in one embodiment, after centrifugation and removal of the supernatant, the alginate gel is dissolved with a chaotropic buffer and the preparation is subsequently brought into contact with a mineral carrier material. Under these conditions the RNA can bind to the mineral material. Optionally, further components can still be added to the chaotropic buffer (alcohols or detergents or mixtures of alcohols and detergents) which can reinforce an optimization of the binding of the RNA to the mineral carrier material. The bound RNA is then washed and finally detached again from the carrier material. Enrichment of exosomes from a sample and subsequent isolation of the RNA enclosed in the exosomes by means of the method according to the invention from a large volume sample can then normally be made possible already within approximately 30 minutes. Thus, the method according to the invention is much simpler and faster than all other methods in this respect and also much faster than techniques known until now. Thus, the method according to the invention solves the task described in an ideal manner.

The invention is explained more in detail below by means of embodiments wherein the embodiments do not represent a limitation of the method according to the invention.

### Embodiment

Embodiment: Enrichment of Exosomes from a Plasma Sample and Subsequent Isolation of RNA

The enrichment and subsequent isolation of the RNA was made from a plasma sample of 5 ml and is carried out as follows:

1. Addition of 100 μl of an aqueous alginate solution (1%) as well as of 500 μl of a calcium chloride solution (1M). Vortex and incubate at room temperature for 5 minutes.
2. Centrifugation at 4500 rpm for 10 minutes. Remove supernatant entirely.
3. Addition of 5 ml water to the pellet and recentrifugation at 4,500 rpm for 5 minutes. Subsequently the RNA is isolated with a commercial kit for the isolation of RNA (innu-PREP micro RNA Kit; Analytik Jena AG) as follows:
4. Resuspending the pellet with 600 μl Lysis Solution RL. Addition of 20 μl proteinase K and incubation at room temperature for 15 minutes.
5. Transferring the preparation to a DNA Spin Filter Column for removal of contained DNA. Centrifugation at 12,000 rpm for 1 minute. Removal of the DNA Filter Column.
6. Addition of a same volume of isopropanol to the filtrate, mixing of the sample and transfer to an RNA Spin Filter Column. Centrifugation at 12,000 rpm for 1 minute. Discarding of the filtrate.
7. Washing the Spin Filter Column twice with ethanol-containing washing buffers (Washing Solution HS and Washing Solution LS). Centrifugation at 12,000 rpm for 1 minute. Discarding of the filtrate.
8. Drying of the Spin Filter Column by short centrifugation.
9. Placing the Spin Filter Column in a new reaction vessel. Addition of 50 μl RNase free water and elution of RNA from the column.
For proof of successful extraction of cell-free RNA, the isolated RNA was subsequently analyzed on an Agilent Bioanalyzer with a small RNA kit.
The Figure shows an electropherogram from which enrichment of the exosomes results.

1. A method for endching microvesicles (exosornes) from a sample and subsequent extraction/isolation of ingredients contained in said microvesicles, preferably RNA, characterized by the following steps:
a) addition of an aqueous solution of a salt of a polyuronic acid to the sample
b) addition of a substance which induces gel formation/pellet formation of the polyuronic acid
c) mixing of the sample and short incubation
d) centrifugation of the sample and removal of the supernalant
e) dissolving the pellet or the gel piece
f) isolation of the ingredients, preferably RNA, in manner known per se.

2-10 (canceled)
